(19) United States
(12) Patent Application Publication
(10) Pub. No.: US 2024/0062908 A1
(43) Pub. Date: Feb. 22, 2024

(54) SYSTEMS AND METHODS TO EFFECTUATE PRESENTATION OF CUSTOMIZED CONTENT TO SUBJECTS WITHIN INTEGRATED APPLICATIONS

(71) Applicant: Solsten, Inc., St. Paul, MN (US)

(72) Inventor: Joseph Jack Schaeppi, Maple Grove, MN (US)

(21) Appl. No.: 17/820,429

(22) Filed: Aug. 17, 2022

(51) Int. Cl. G16H 50/30

(52) U.S. Cl. CPC G16H 50/30

(57) ABSTRACT

Systems and methods to effectuate presentation of customized content to subjects within integrated applications are disclosed. Exemplary implementations may: effectuate presentation of a set of pieces of content within digital environments via integrated applications interacted with by a subject to fulfill an objective related to the subject; receive interaction information from the integrated applications related to the subject and the pieces of content; determine, based on the interaction information, impact values of the pieces of content in the set on the objective for the subject over a period of time; determine, based on the impact values, correlations between the pieces of content in the set with aspects of the objective; and adjust, based on the correlations, the digital environments of the integrated applications to include the pieces of content from the set that facilitate fulfillment of the objective and remove the pieces of content from the set that do not.

### FIELD OF THE DISCLOSURE

The present disclosure relates to systems and methods to effectuate presentation of customized content to subjects within integrated applications.

### BACKGROUND

Subjects typically use a plurality of applications via smart devices and thus interact with a plurality of various content provided by those applications. Healthcare providers and/or caregivers may seek to assess subjects and/or provide treatments. However, in-person or virtual appointments may be demanding and/or require subjects to interact with an additional application for assessments and/or treatments may be ineffective. Furthermore, popular applications amongst the subjects may not have the capabilities to analyze the subjects' interactions with the applications to generate assessments nor provide treatments.

### SUMMARY

The present disclosure may be related to customizing content provided by various applications integrated with a system described herein. A set of pieces of content may be presented within digital environments via the integrated applications to fulfill an objective. For example, the objective may be to determine health assessment information or provide treatment and/or guidance via the set of pieces of content. Interaction information from the integrated applications may be obtained. Based on the interaction information, impact values for the individual pieces of content in the set may be determined. The impact values may be related to the objective. Based on the impact values, correlations between the individual pieces of content and aspects of the objective may be determined. Based on the correlations, the digital environments may be adjusted to include the pieces of content that facilitate fulfillment of the objective and remove the pieces of content that do not facilitate fulfillment of the objective. Thus, the individual integrated applications are not required to determine techniques to accomplish the objective and the digital environments of the integrated applications are seamlessly adjusted and customized for the subjects as determined appropriate.

One aspect of the present disclosure relates to a system configured to effectuate presentation of customized content to subjects within integrated applications. The system may include one or more hardware processors configured by machine-readable instructions, electronic storage, and/or other components. Machine-readable instructions 106 may include one or more instruction components. The instruction components may include computer program components. The instruction components may include one or more of presentation component, information receiving component, impact determination component, correlation determination component, environment adjusting component, and/or other instruction components.

The electronic storage may be configured to store taxonomical classifications of individual pieces of content, psychological profiles for subjects, health assessment information of the subjects, and/or other information. Individual taxonomical classifications may include content parameter values for content parameters that define classifications for the individual pieces of content. The taxonomical classifications may conform to a taxonomy that defines a hierarchical system of the content parameters that facilitate providing the pieces of content with the classifications. The pieces of content may be defined by the content parameter values for some or all of the content parameters. The psychological profiles may include psychological parameter values for psychological parameters.

The presentation component may be configured to effectuate presentation of a set of the pieces of content within digital environments via integrated applications interacted with by the subject to fulfill an objective related to the subject.

The information receiving component may be configured to receive interaction information from the integrated applications and/or other information. The interaction information may characterize interactions between the subject and the set of the pieces of content.

The impact determination component may be configured to determine, based on the interaction information and/or other information, impact values of the pieces of content in the set on the objective for the subject over a period of time.

The correlation determination component may be configured to determine, based on the impact values, correlations between the pieces of content in the set with aspects of the objective. The correlations may include the corresponding impact values.

The environment adjusting component may be configured to adjust, based on the correlations, the digital environments of the integrated applications to regularly include the pieces of content from the set that positively impacted the objective and remove the pieces of content from the set that negatively impacted the objective.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to effectuate presentation of customized content to subjects within integrated applications, in accordance with one or more implementations.

FIG. 2 illustrates a method to effectuate presentation of customized content to subjects within integrated applications, in accordance with one or more implementations.

FIG. 3 illustrates an example implementation of the system configured to effectuate presentation of customized content to subjects within integrated applications, in accordance with one or more implementations.

### DETAILED DESCRIPTION

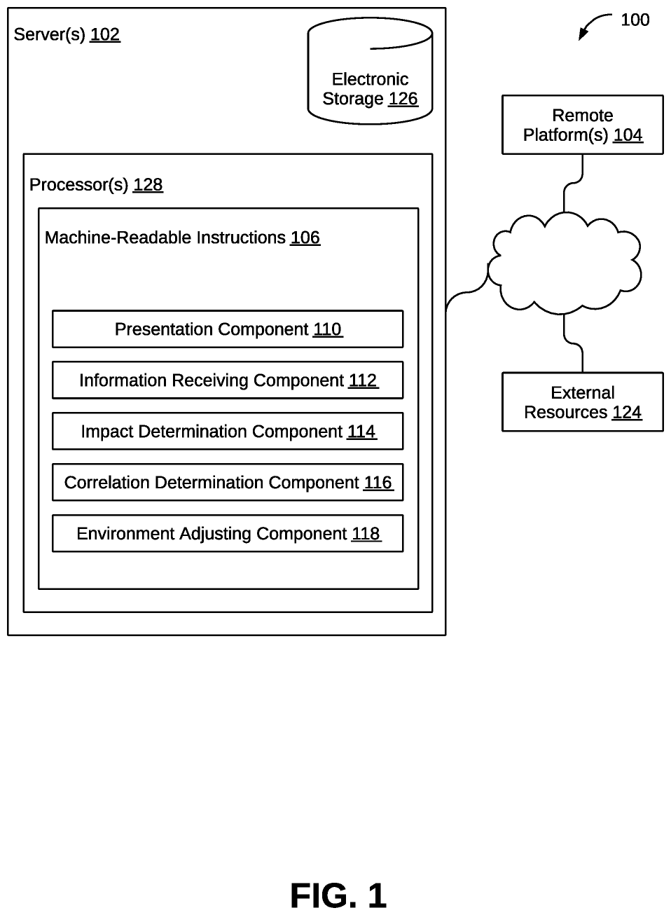

FIG. 1 illustrates a system 100 configured to effectuate presentation of customized content to subjects within integrated applications, in accordance with one or more implementations. In some implementations, system 100 may include one or more servers 102. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may be configured by machine-readable instructions 106. Machine-readable instructions 106 may include one or more instruction components. The instruction components may include computer program components. The instruction components may include one or more of presentation component 110, information receiving component 112, impact determination component 114, correlation determination component 116, environment adjusting component 118, and/or other instruction components.

Electronic storage 126 may be configured to store taxonomical classifications of individual pieces of content, psychological profiles for subjects, health assessment information of the subjects, and/or other information. Individual taxonomical classifications may include content parameter values for content parameters that define classifications for the individual pieces of content. The subjects may be patients or people who require assistance. The taxonomical classifications may conform to a taxonomy that defines a hierarchical system of the content parameters that facilitate providing the pieces of content with the classifications.

The pieces of content may be defined by the content parameter values for some or all of the content parameters. The pieces of content may include a character, a game, a game asset, a recommendation, a suggestion, a promotion, and/or other pieces of content. The character may refer to an object (or group of objects) present in a virtual space that corresponds to an individual subject (e.g., an avatar) and/or are controlled by the subject. In some implementations, the character may not correspond to an individual subject but rather provide information (e.g., the recommendation, the suggestion) to the subject. The game asset may include a virtual item, a virtual resource (e.g., weapon, tool), of in-game powers, in-game skills, in-game technologies, and/or other game assets. The recommendation may include a particular selection and/or action that the subject is advised to select and/or do. The suggestion may include particular ideas, plans, and/or strategies for the subject to consider executing, following, and/or is determined they will enjoy. The promotion may include discount codes, coupons, bonuses, and/or other promotions of the virtual items, products, and/or services that the subject may utilize. By way of non-limiting example, products and/or services may relate to beauty (e.g., skincare, makeup), home improvement, decoration, clothing, accessories, technology, kitchen, and/or other categories.

Individual taxonomical classifications may include content parameter values for content parameters that define classifications for the individual pieces of content. The taxonomical classifications may conform to a taxonomy that defines a hierarchical system of the content parameters that facilitate providing the pieces of content with the classifications. The pieces of content may be defined by the content parameter values for some or all of the content parameters.

Some of the content parameters may be high order parameters and some of the content parameters may be lower order parameters. That is, a high order parameter may include more specific lower order parameters where the content parameter values more specifically describe the content as the content parameter values exist for the lower order parameters. In some implementations, a parameter may be one or more hierarchical orders within the taxonomy. The content parameters may include genre, platform-specific genre, mechanics, theme, art style and perspective, brand intellectual property, modes, churn, marketing assets, creative elements, and/or other content parameters. Such content parameters may be the higher order parameters of the content parameters. The higher order parameters may include other content parameters as described in co-pending U.S. application Ser. No. 17/592,257 entitled “SYSTEMS AND METHODS TO IDENTIFY A TARGET AUDIENCE FOR PROSPECTIVE CONTENT BASED ON A TAXONOMY”, Attorney Docket No.

The psychological profiles may include psychological parameter values for psychological parameters. The psychological profiles may include sets of psychological parameter values to the psychological parameters for the individual subjects. By way of non-limiting example, the psychological parameter values of the psychological parameters may be a number score on a predetermined range unique to each psychological parameter, a letter score, and/or other type of value than may characterize a particular subject as whole.

Parameters, such as psychological parameters are described herein, may specify measurable, recordable, and/or determined information. The parameter values corresponding to the parameters may be a particular value, numerical or non-numerical, that characterizes the content, the users, or respective element that the parameter value is described in relation to.

The psychological parameter values may characterize, by way of non-limiting example, achievement motivation, motivation, personality inventory, cultural values, competitiveness, positive and negative affect before, during, and/or after engagement with the digital experience (i.e., emotions), communication style, personal values, daily routines/activities, life/gaming pain points, life/gaming hopes and aspirations, wellbeing, user experience, gaming/experience using time, subscription behavior, affinity information, personality, emotional style, goal orientation, goal commitment, ego and task orientation, relatedness, sense of community, social influence, social identity, group identification, we-identity, quality of life, satisfaction with life, work-related quality of life, mindfulness, happiness, emotional intelligence, self-awareness/internal awareness, external awareness, connectedness to nature, social connectedness, social bonding, perceived stress, depression, anxiety, decision-making style, thinking style, critical thinking, cognitive approach to learning, learning style, attributional style, internality-externality, stability-instability, global-specific, creativity, curiosity, playfulness, exploration, mental strength, grit, flourishing, gratitude, inspiration, spirituality, hedonism, materialism/material values, perceptions, sentiments, and/or other psychological parameters.

Achievement motivation may include compensatory effort, competitiveness, confidence in success, dominance, eagerness to learn, engagement, fearlessness, flexibility, flow, goal setting, independence, internality, persistence, preference in difficult tasks, pride in productivity, self-control, status orientation, ambition, self-assurance, and/or other psychological parameters. Motivation may include mastery, purpose, autonomy, and/or other psychological parameters.

Personality inventory may include neuroticism, openness, conscientiousness, extraversion, and agreeableness and/or other psychological parameters. Neuroticism may include anxiety, impulsiveness, vulnerability, and/or other psychological parameters. Openness may include fantasy, feelings/empathy, action, and/or other psychological parameters. Conscientiousness may include achievement striving, competence, self-discipline, and/or other psychological parameters. Extraversion may include warmth assertiveness, activity, and/or other psychological parameters. Agreeableness may include trust, altruism, modesty, and/or other psychological parameters.

Cultural values may include individualism, indulgence, long term orientation, masculinity, power distance, uncertainty avoidance, and/or other psychological parameters. Competitiveness may include avoidant, collaborative, competitive affectivity, dependent, dominant, general competitiveness, independent, personal enhancement, and/or other psychological parameters.

Positive and negative affect before, during, and/or after engaging in the digital experience may include hostility, joviality, negative emotions, positive emotions, sadness, self-assurance, and/or other psychological parameters. Communication style may include feeler, intuitor, sensor, thinker, and/or other psychological parameters.

Wellbeing may include social wellbeing, psychological wellbeing, physical wellbeing, physical activity, sleep, bounded reciprocity, resilience grit, and/or other psychological parameters.

Personality may include anger, hostility, depression, self-conscientiousness, excitement-seeking, positive emotions, gregariousness, ideas, values, aesthetics, tender-mindedness, straightforwardness, compliance, deliberation, order, dutifulness, and/or other psychological parameters.

Emotional style may include resilience, outlook, social intuition, self-awareness, sensitivity to context, attention, and/or other psychological parameters.

Goal orientation may include mastery approach/learning goal orientation, performance approach/performance goal orientation, performance avoid/avoidance goal orientation, and/or other psychological parameters.

Work-related quality of life may include structure, boundaries, focus, efficiency, information provision, communication, psychological support, stress at/from work, psychological safety, connectedness with team, motivation to work, adaptability, job/career satisfaction, control at work, home-work interface, general wellbeing, working conditions, and/or other psychological parameters.

Mindfulness may include observing, describing, acting with awareness, non-judgment, non-reactivity, and/or other psychological parameters.

Emotional intelligence may include emotion perception, emotion expression, emotion management, emotion regulation, impulse control, relationships, stress management, and/or other psychological parameters.

Social connectedness may include social connectedness, loneliness, membership self-esteem, private self-esteem, public self-esteem, identity self-esteem, interdependent self, independent self, social avoidance, social distress, and/or other psychological parameters.

Decision-making style may include respected, confident, spontaneous, dependent, vigilant, avoidant, brooding, intuitive, anxious, and/or other psychological parameters.

Thinking style may include intuitive, experiential, analytical, rational, and/or other psychological parameters.

Cognitive approaches to learning may include avoidant, participative, competitive, collaborative, dependent, independent, and/or other psychological parameters.

Learning style may include visual (spatial), aural (auditory-musical), verbal (linguistic), physical (kinesthetic), logical (mathematical), social (interpersonal), solitary (intrapersonal), and/or other psychological parameters.

Mental strength may include tenacity, confidence, optimism, adaptability, self-awareness, reliability, responsibility, well-being, and/or other psychological parameters.

Flourishing may include positive emotion, engagement, relationships, meaning, accomplishment, health, loneliness, and/or other psychological parameters.

The health assessment information may include a current mental state, a current emotional state, changes for the psychological parameter values to the psychological parameters, that comprise the psychological profile and/or other health assessment information. The health assessment information may indicate new disorders, illnesses, and/or conditions of the subject. In some implementations, the health assessment information may indicate changes in identified disorders, illnesses, and/or conditions of the subject. In some implementations, the health assessment information may indicate probable or impeding disorders, illnesses, and/or conditions of the subject. By way of non-limiting example, the disorders, illnesses, and/or conditions may include insomnia, eating disorders, addictions, mood disorders, learning disabilities (e.g., dyslexia, dyscalculia, dysgraphia, etc.), personality disorders, psychotic disorders, and/or other disorders, illnesses, and/or conditions.

The current mental state may be how the subject processes and/or understands their thoughts and actions. The current mental state may include one or more of psychological well-being, social well-being, and/or emotional well-being, and/or other well-being of the subject, collectively. The current mental state may be reflected or represented in one or more of the subject's sleeping patterns (e.g., not enough sleep, excessive sleeping, 8-hour sleep, restlessness), eating patterns (e.g., overeating, under-eating, purging), energy, mood, substance consumption, and/or other representations.

Some or all of the psychological parameter values to the psychological parameters may define, closely define, or facilitate determining the psychological well-being, the social well-being, and the emotional well-being, collectively, of the subject. In some implementations, sets of the psychological parameters and their psychological parameter values for the subject and particular interactions with the content and/or specific content during the session may characterize the representations of the current mental state and thus the psychological well-being, the social well-being, and the emotional well-being of the subject. For example, a first set of psychological parameters and the psychological parameter values thereof, and first content interacted with during the session may characterize the sleeping pattern of the subject and indicate that the subject has insomnia. Additionally, the application usage information, such as the screen time, may additionally indicate that the subject has insomnia, cognition of the subject, mental health characteristics, and/or other disorders, illnesses, and/or conditions. By way of non-limiting example, the mental health characteristics may include inability to deal with stress, violence, hostility, anger, trouble relating to others or situations, low energy, inability to concentrate, and/or other mental health characteristics.

The current emotional state may be having awareness of emotions and having the ability to manage and express the emotions appropriately, or lack thereof. The current emotional state may be reflected or represented in one or more of the subject's relationships with people (e.g., friends, family, authority, strangers), communication with the people, reactions to receiving communications from the people, reactions to events or circumstances (e.g., failing a test, accomplishing a goal), and/or other representations. In some implementations, some of the psychological parameter values to one or more of the psychological parameters related to the emotional intelligence, the emotional style, the flourishing psychological parameters, and/or other psychological parameters may particularly facilitate determination of the current emotional state in addition to session usage information described herein, the application usage information, interaction information, and/or other information.

In some implementations, stated information obtained by information receiving component 112 may include acknowledgements of particular ones of the conditions, illnesses, disorders, and/or other information. The current mental state and the current emotional state may affect the conditions, illnesses, disorders, severity thereof, and how such present themselves by way of the subject. Based on the acknowledgements, determination of the health assessment information may be focused on aspects of the psychological profile, the session usage information, and/or other information that are related to those acknowledgements or the aspects that otherwise indicate the acknowledged conditions, illnesses, disorders by way of one or more particular pieces of content.

In some implementations, the current mental state and/or the current emotional state may be quantified or represented via the psychological profile. Thus, the determined changes to the psychological profile of the subject (i.e., the psychological parameter values to one or more of the psychological parameters) may be based on the session usage information, the acknowledgements, a past psychological profile (i.e., previously determine psychological parameter values), and/or the other information described herein. Such changes may be presented and/or updated upon obtainment and/or determination.

Presentation component 110 may be configured to effectuate presentation of a set of the pieces of content within digital environments via integrated applications interacted with by the subject to fulfill an objective related to the subject.

The integrated applications may be provided by and/or hosted by online platforms. The integrated applications may be accessed online or offline by the subjects. The integrated applications may synchronize and/or communicate with information receiving component 112 upon an online connection. The communication may be facilitated by a network and/or the Internet. The integrated applications, and application types thereof, may include, By way of non-limiting example, game applications, educational applications, reading applications, music applications, social networking applications, entertainment applications, fitness applications, business applications, shopping applications, food & drink applications, among other applications integrated with system 100.

Client computing platforms 104 (e.g., smartphone, laptop, game console) associated with the subjects may provide access to a plurality of the applications. In some implementations, the application provided by client computing platform 104 may be initially provided by online platforms (e.g., downloaded from the online platforms). In some implementations, an online platform and a digital environment provided thereof may be referred to as the application. As used herein, the terms “integrated application”, “online application”, “application”, and variations thereof may be used interchangeably and refer to digital environments provided by online platforms that the subjects may interact with online or offline.

The digital environment and/or individual applications may provide simulated spaces or views of a virtual space. Individual simulated spaces may have a topography, express ongoing real-time interaction by one or more subjects, and/or include one or more objects positioned within the topography that are capable of locomotion within the topography. In some instances, the topography may be a 2-dimensional topography. In other instances, the topography may be a 3-dimensional topography. The topography may include dimensions of the space, and/or surface features of a surface or objects that are “native” to the space. In some instances, the topography may describe a surface (e.g., a ground surface) that runs through at least a substantial section of the space. In some instances, the topography may describe a volume with one or more bodies positioned therein (e.g., a simulation of gravity-deprived space with one or more celestial bodies positioned therein). The instance executed by the computer components may be synchronous, asynchronous, and/or semi-synchronous.

The presentation of the set of the pieces of content within digital environments via integrated applications may be to fulfill some objective related to the subject. In some implementations, the objective may be initiated responsive to receipt of the objective. The objective may be received by information receiving component 112 from client computing platforms 104 associated with the subject themselves, a caregiver, and/or other user or subject. The caregiver may include a doctor, a therapist, a nurse, a mental health provider (e.g., psychologist), a guardian, a parent, and/or other caregiver. In some implementations, the objective may be prescheduled and/or reoccurring. For example, the caregiver may define a first objective to be fulfilled every month, and a second objective to be fulfilled every two weeks.

In some implementations, the objective related to the subject may be determination of the health assessment information. In some implementations, some of the pieces of content in the set presented to the subject to determine the health assessment information may prompt a response from the given subject. Such pieces of content may include timed challenges, written response questions, riddles, visual games, auditory games, memory games, and/or other content requiring responses. In some implementations, individual pieces of content in the set may correlate with the current mental state of the given subject, the current emotional state of the given subject, and/or changes in the psychological profile of the given subject. Such correlations may facilitate determining the respective aspects of the health assessment information. That is, the responses received responsive to the correlated pieces of content may be indicative of changes in the respective aspects of the health assessment information. The correlations between the pieces of content and different aspects of the health assessment information may be determined as described herein, and/or may be predefined by a user.

In some implementations, the objective may be to provide treatment and/or guidance to affect the health assessment information responsive to the health assessment information. By way of non-limiting example, the treatment may include physical activity, medication, therapy, focusing activities, meditation, utilizing particular devices (e.g., sound machine, blue lights, red lights), and/or other treatments. By way of non-limiting example, the guidance may include messages of comfort, messages of direction related to the treatment, messages of encouragement, testimonies, reminders related to the treatment, tips related to the treatment, and/or other guidance.

Information receiving component 112 may be configured to receive interaction information, application usage information, the session usage information, and/or other information from the integrated applications or from other sources. The interaction information may characterize interactions between the subject and the set of the pieces of content. The interaction information may include the content engaged with by the subjects, how the individual subjects engaged with the content, and/or other interaction information. The content engaged with by the individual subjects may be related to the online platforms or the individual online applications that provide the content. That is, for example, the content provided by an online platform may relate to online games (e.g., virtual goods, virtual mini games, etc.). In some implementations, the content engaged with by the individual subjects may not be related to the online platforms that provide the content. Meaning, the content may direct the subject to a different online application or online platform.

How the subjects engage with the content, or engagement by the individual subjects, may define behavior patterns of the individual subjects with or based on the content. The behavior patterns of the individual subjects may include individual actions, sets of actions, ordered sets of actions, time spent by the individual subjects engaging with the content and/or the online platforms, spending patterns of the subjects, completed tasks by the individual subjects, uncompletion tasks by the individual subjects, failure of tasks by the individual subjects, game mechanics initiated by the subjects, and/or other behavior patterns. In some implementations, the behavior patterns may include multiple of the individual actions, the sets of actions, and the ordered set of actions. The actions may include one or more of a purchase based on the content, a sale, a trade, a donation, a subject selection of the content, gameplay (e.g., mini-game, battle, competition, etc.) based on the content, communication of the individual subjects with other particular subjects or users, completion of tasks by the subjects, frequent interaction with the content, formation of alliances by the subjects, and/or other actions. The spending patterns may indicate an amount of currency (e.g., real-world money, virtual money, points, etc.) spend, an amount of currency earned, an amount of currency donated, and/or other indications.

In some implementations, the interactions of the subjects with the digital environments and/or the behavior patterns of the subjects may be correlated with psychological attributes, i.e., the psychological parameter values to the psychological parameters, as described in U.S. application Ser. No. 16/894,522 entitled “SYSTEMS AND METHODS TO CORRELATE USER BEHAVIOR PATTERNS WITHIN AN ONLINE GAME WITH PSYCHOLOGICAL ATTRIBUTES OF USERS” Attorney Docket No. 01TT-064002, the disclosure of which is incorporated by reference in its entirety herein.

The application usage information may include values to screen time on the individual applications, battery usage of the individual applications, Internet usage of the individual applications, location usage by the individual applications, times of installations of the individual applications, the application types of installations of the individual applications, costs of the installations, times of initiations of the individual applications, times of terminations of the individual applications, amount of notifications from the individual applications, notification types of the notifications, response to the notifications, cross-application information usage, times of in-application purchases and in-application sales, item type of the in-application purchases, the item type of the in-application sales, the application types of the applications initiated, and/or other usage parameters.

The screen time and/or battery usage may indicate an amount of time that the subject spends on client computing platform 104 collectively over a period of time and/or an amount of time the subject spends on individual applications. The Internet usage may indicate an amount of time and when the subject is online (i.e., using the Internet) and offline within the applications. The location usage may indicate how often a location of client computing platform 104 is requested of the subject and/or authorized for use by the subject. The location usage may indicate where the subject is during use of the online applications and/or movement during the use of the online applications (e.g., travelling by car, static at work). The response to the notifications and the notification types thereof may indicate what types of notifications the subject often responds to and ignores. The notification types may include, by way of non-limiting example, a time sensitive notification (e.g., a sale countdown for a virtual or physical item), a question notification, a feedback request (e.g., experience survey), a contest or sweepstakes entry, an incentive (e.g., if you provide feedback, you get a gift card), a reminder, informational (e.g., weather alert), a promotion/deal, and/or other notification types.

The session usage information may characterize current use of the online application by the subject. The current use of individual online applications may refer to how the subject is interacting with the online application and content thereof, when the subject is interacting or using the online application, what content the subject is interacting with, what other subjects or people they subject is communicating or interacting with via the online application, and/or other uses during a particular time range. The time range may be from a first date to a second date, a particular time span of a day (e.g., 6 PM-10 PM), the particular time span of a day over multiple days, the particular time span of a day on particular days of the week, the particular time span of a day on specific dates, and/or other time ranges.

The current use may be a particular time period during which the subject is being evaluated, monitored, measured, or otherwise examined via their use of the online applications. Such session usage information may be utilized by medical professionals (e.g., physicians, psychologists, psychiatrists), caregivers, and/or others to provide healthcare.

The session usage information may include game mechanics within the online application the subject employed, the game mechanics within the online application the subject avoided, one or more games chosen to be played, one or more of the games disregarded, session results, read literature, unread literature, social interactions, and/or other session usage information.

The game mechanics may include alternating turns in the one or more games, action points, playing cards, capturing, catch-up progression, dice, movements, resource management, risk and reward, role-playing, game modes (e.g., single-player, multiplayer), and/or other novel or known game mechanics. In some implementations, different ones of the games and/or the content provided by the online application may employ different game mechanics. Thus, the subject may initiate and utilize different game mechanics within the online application. Conversely, the subject may disregard some of the game mechanics by disregarding some of the games and/or the content within the online application.

The session results may include wins, losses, virtual items acquired, virtual items dropped, level attempts, levels achieved, characters played as, characters battled and subjects thereof, in-application or in-game movements, and/or other session results. The wins and losses may be of the one or more games, and/or other wins and losses. The virtual items acquired and dropped may be from the one or more games won and lost, respectively. For example, the virtual items may include tokens, stars, digital currency, virtual goods (e.g., weapons, tools, food, clothing, etc.), and/or other virtual items. The in-application or in-game movements may include tracked movements around a map of a game, movements along a game board, movements of playing cards (e.g., Solitaire), action or battle movements (e.g., jump-shoot, flip and kick, sword combinations, punch combinations), and/or other movements. The games within the online applications may be mini-games within an encompassing game provided by the online application, distinct mini-games, and/or other games. The games may have a game type. For example, the game types may include a puzzle game, a matching game, a racing game, a strategy game, a role-playing game, a party game, a multiplayer battle game, an action-adventure game, a survival and horror game, a sports simulation game, a shooter game, and/or other game types.

The literature may include manuals, books, articles, policies, messages, law, and/or other literature. The social interactions may include direct messages between the subject and other users or subjects, audio communication between one or more other users and/or subjects, video communications between the subject and one or more other users and/or subjects, public messages between the subject and one or more other users and/or subjects (e.g., comments under a post), selecting a virtual button that indicates a reaction (e.g., like, dislike, agreement, disagreement, laughter), and/or other social interactions.

Information receiving component 112 may be configured to store the interaction information, the application usage information, the session information, and/or other information received to electronic storage 126 and in associated with the appropriate subject. In some implementations, electronic storage 126 may be organized to store subject summaries for the subjects. An individual subject summary may include identifying information, the psychological profile of a given subject, the application usage information, the interaction information, past session usage information, treatments and/or guidance, and/or other information determined about or for the given subject. The identifying information may include a name, a date of birth, an address, a phone number, an email address, a medical record number, and/or other information. In some implementations, the identifying information may not be stored in association with a specific subject.

Impact determination component 114 may be configured to determine impact values of the pieces of content in the set on the objective for the subject over a period of time. The impact values may be determined based on the psychological profile of the given subject, the application usage information, the interaction information, past session usage information, the treatments and/or guidance, and/or other information. In some implementations, the impact values may be a value in accordance with an impact value structure such as a predefined range, a letter score, a deviation from a neutral or center value, a binary value (e.g., positive or negative), and/or other impact value structure. In some implementations, the impact value structure may be selected and/or changed by the caregiver. In some implementations, the impact value structure may be predefined.

In some implementations where the objective is determining the health assessment information, determining the impact values may include determining frequent interactions with the pieces of content in the set and determining whether the pieces of content affect the determination of the health assessment information. The frequent interactions may be determined based on the interaction information and/or other information received by the information receiving component 112. The frequent interactions with the pieces of content may include more than a minimum amount of interactions over a period of time. In some implementations, the frequent interaction may be of one or more particular interactions with the pieces of content. In some implementations, determining the impact values may be based on machine learning, artificial intelligence analysis, and/or other techniques. As such, it may be determined that some of the pieces of content in the set facilitate with determination of the health assessment information, and others of the pieces of content in the set do not facilitate with determination of the health assessment information. Such effectiveness may be represented as the impact values.

In some implementations where the objective is to provide the treatment and/or guidance to affect the health assessment information, the pieces of content in the set that may present the treatment and/or guidance. The pieces of content in the set that present the treatment and/or guidance may facilitate the subject with compliance of the treatment, determination of effectiveness of the treatment and/or guidance, determination of new treatments and/or guidance, and/or other information related to the treatment and/or guidance. The compliance may include the subject completing the treatment and/or attempting to complete the treatment. The determination of the effectiveness of the treatment and/or guidance may include determination of whether the treatment caused desired or undesired changes in the health assessment information and/or whether the guidance contributed to the compliance. The determination of the new treatments and/or guidance may be based on the treatment and/or guidance currently being presented by the pieces of content in the set.

Determining the impact values may include determining frequent interactions with the pieces of content in the set and determining positive and/or negative changes to the health assessment information. That is, for example, a first treatment may be presented via a first piece of content and a different second piece of content. It may be determined that the first treatment presented as the first piece of content affected the health assessment information (e.g., emotional health improved) because the subject completed the first treatment. It may be determined that the first treatment presented as the second piece of content affected the health assessment information (e.g., emotional health declined) because the subject did not complete the first treatment. Such effectiveness may be represented as the impact values for the individual pieces of content (e.g., the first piece and the second piece).

Correlation determination component 116 may be configured to determine, based on the impact values and/or other information, correlations between the pieces of content in the set with aspects of the objective. In some implementations, the correlations determined may be between the content parameter values to the content parameters for the individual pieces of the content in set and the aspects of the objective. Thus, the correlations may be between an entire classification of content and the aspects of the objective.

In some implementations where the objective is to determine the health assessment information, the aspects of the objective may include the current mental state, the current emotional state, individual psychological parameters, and/or other aspects of the health assessment information. Thus, individual pieces of content may be correlated with one or more of the current mental state, the current emotional state, individual psychological parameters, and/or other aspects of the health assessment information. It will be appreciated that the description herein of “correlations” between individual pieces of content and aspects of the objective which are positively correlated is not intended to be limiting, and that negative correlations between individual pieces of content and aspects of the objective, for example, are also contemplated, and may be included in the generic “correlations”. The determination of negative correlations may be made in cases where a piece of content that the subject frequently interacted with did not facilitate with determination of any aspects of the health assessment information, for example. In some implementations, the correlations may include or be associated with the corresponding impact values.

In some implementations where the objective is to provide the treatment and/or guidance to affect the health assessment information, the aspects of the objective may include changes, positive or negative, to the health assessment information. To determine whether the health assessment information changed responsive to the treatment and/or guidance, new or current health assessment information may be determined as described in co-pending U.S. application Ser. No. 17/888,218 entitled “SYSTEMS AND METHODS TO ASSESS HEALTH INFORMATION FOR A SUBJECT BASED ON SUBJECT-INITIATED SESSIONS WITH ONLINE APPLICATIONS”, Attorney Docket No. 01TT-064016. The individual pieces of content may be correlated with changes in one or more aspects of the health assessment information. Thus, individual pieces of content that present the treatment and/or guidance may be correlated with a positive change to the health assessment information (e.g., mental state improved) and/or a negative change to the health assessment information (e.g., psychological parameter values negatively changed).

Environment adjusting component 118 may be configured to adjust, based on the correlations and/or other information, the digital environments of the integrated applications to regularly include the pieces of content from the set that positively impacted the objective and remove the pieces of content from the set that negatively impacted the objective. Regularly including the pieces of content may refer to the digital environments including the pieces of content from the set that positively impacted the objective every time the subject interacts with the digital environments, presenting new pieces of content that are similar recurringly after an amount of time as passed (e.g., every week), including content that is similar to the pieces of content from the set every time the subject interacts with the digital environments, presenting new pieces of content that are similar recurringly after an amount of sessions or interactions have occurred (e.g., after every 5 interactions with the digital environments), and/or other reoccurrences for frequent presentation to the subjects.

A positive impact on the objective may refer to facilitating fulfillment of the objective. For example, a first piece of content that has a positive impact may be that the first piece of content facilitated determination all or some aspects of the health assessment information. A negative impact on the objective may refer to not facilitating fulfillment of the objective. For example, a second piece of content that has a negative impact may be that the second piece of content did not facilitate with determination of the effectiveness of the treatment and/or guidance. As such, the first piece of content may regularly be included in the digital environments and the second piece of content may be removed from regular inclusion or inclusion all together in the digital environments. Thus, providers and/or developers of the integrated applications need not determine nor conduct determinations of whether the content presented to individual subjects is affective the subjects, how it is affecting the subjects, and changed presentation of such based on the effect. Rather, the integrated applications may provide the interaction information and/or other information and subsequently be adjusted as appropriate for each subject.

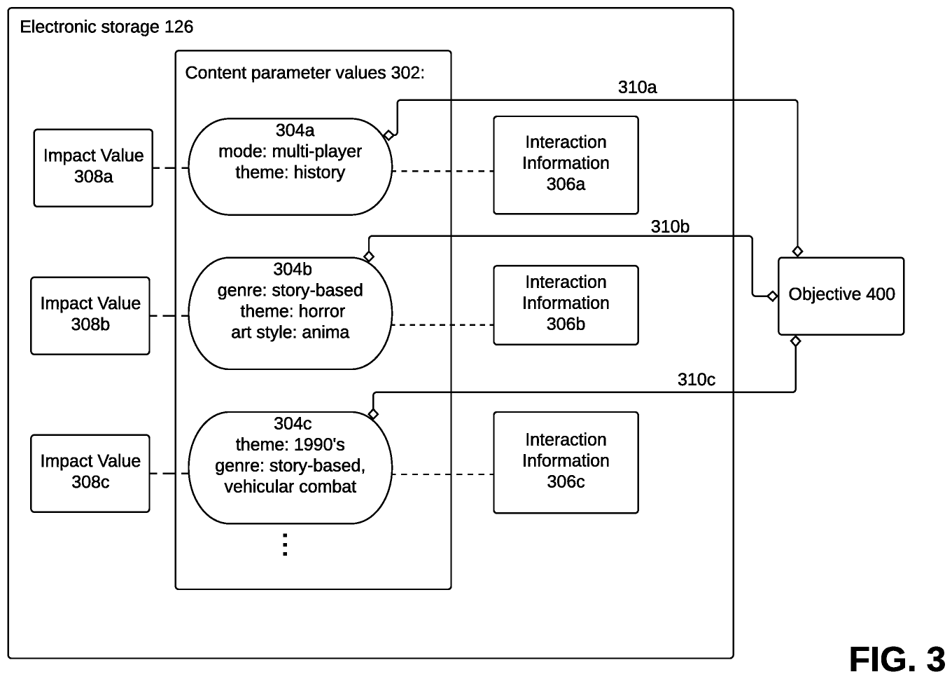

FIG. 3 illustrates an example implementation. Electronic storage 126 (as illustrated in FIG. 1) may store pieces of content 304a, 304b, and 304c. Each of pieces of content 304a, 304b, and 304c may be defined by content parameter values 302 to content parameters. Pieces of content 304a, 304b, and 304c may be presented via digital environments provided by integrated applications to fulfill an objective 400. Based on pieces of content 304a, 304b, and 304c presented, related interaction information 306a, 306b, and 306c, respectively, may be obtained from the integrated applications. Impact values 308a, 308b, and 308c for pieces of content 304a, 304b, and 304c, respectively, may be determined based on interaction information 306a, 306b, and 306c, respectively. Correlations 310a, 310b, and 310c between individual pieces of content 304a, 304b, and 304c and objective 400, or aspects thereof, may be determined based on impact values 308a, 308b, and 308c. Correlations 310a, 310b, and 310c may indicate whether pieces of content 304a, 304b, and 304c, respectively, facilitate fulfillment of objective 400 or not. Thus, the digital environments may be adjusted, based on correlations 310a, 310b, and 310c, to include the pieces of content that facilitate fulfillment of objective 400 and remove the pieces of content that do not facilitate fulfillment of objective 400.

In some implementations, server(s) 102, client computing platform(s) 104, and/or external resources 124 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 124 may be operatively linked via some other communication media.

A given client computing platform 104 may include one or more processors configured to execute computer program components. The computer program components may be configured to enable an expert or user associated with the given client computing platform 104 to interface with system 100 and/or external resources 124, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 124 may include sources of information outside of system 100, external entities participating with system 100, and/or other resources. In some implementations, some or all of the functionality attributed herein to external resources 124 may be provided by resources included in system 100.

Server(s) 102 may include electronic storage 126, one or more processors 128, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 126 may comprise non-transitory storage media that electronically stores information. The electronic storage media of electronic storage 126 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 126 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 126 may include one or more virtual storage resources (e.g., cloud storage, a virtual private network, and/or other virtual storage resources). Electronic storage 126 may store software algorithms, information determined by processor(s) 128, information received from server(s) 102, information received from client computing platform(s) 104, and/or other information that enables server(s) 102 to function as described herein.

Processor(s) 128 may be configured to provide information processing capabilities in server(s) 102. As such, processor(s) 128 may include one or more of a digital processor, an analog processor, a digital circuit designed to process information, an analog circuit designed to process information, a state machine, and/or other mechanisms for electronically processing information. Although processor(s) 128 is shown in FIG. 1 as a single entity, this is for illustrative purposes only. In some implementations, processor(s) 128 may include a plurality of processing units. These processing units may be physically located within the same device, or processor(s) 128 may represent processing functionality of a plurality of devices operating in coordination. Processor(s) 128 may be configured to execute components 110, 112, 114, 116, and/or 118, and/or other components. Processor(s) 128 may be configured to execute components 110, 112, 114, 116, and/or 118, and/or other components by software; hardware; firmware; some combination of software, hardware, and/or firmware; and/or other mechanisms for configuring processing capabilities on processor(s) 128. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 110, 112, 114, 116, and/or 118 are illustrated in FIG. 1 as being implemented within a single processing unit, in implementations in which processor(s) 128 includes multiple processing units, one or more of components 110, 112, 114, 116, and/or 118 may be implemented remotely from the other components. The description of the functionality provided by the different components 110, 112, 114, 116, and/or 118 described below is for illustrative purposes, and is not intended to be limiting, as any of components 110, 112, 114, 116, and/or 118 may provide more or less functionality than is described. For example, one or more of components 110, 112, 114, 116, and/or 118 may be eliminated, and some or all of its functionality may be provided by other ones of components 110, 112, 114, 116, and/or 118. As another example, processor(s) 128 may be configured to execute one or more additional components that may perform some or all of the functionality attributed below to one of components 110, 112, 114, 116, and/or 118.

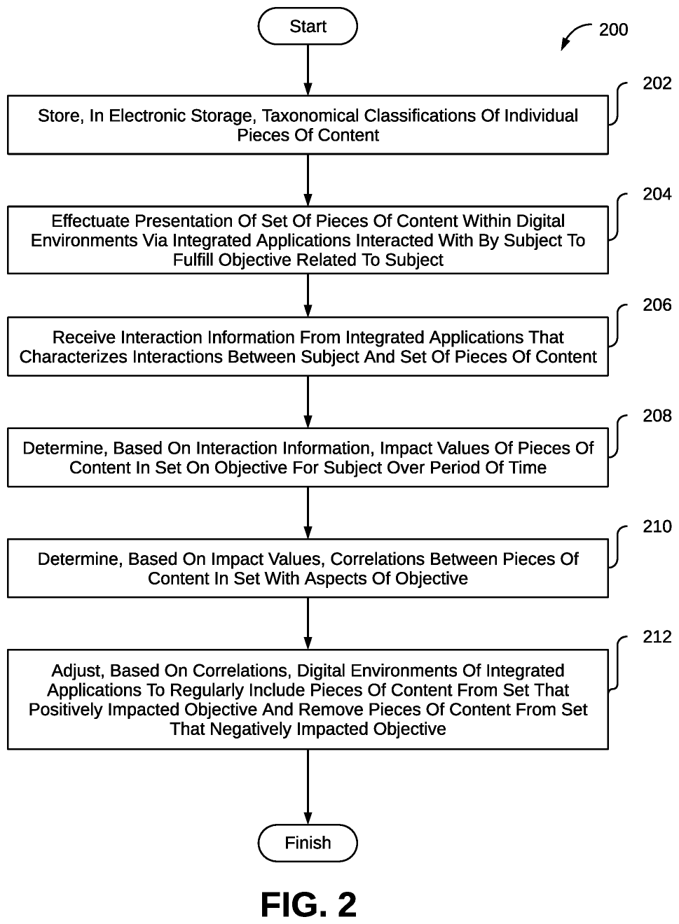

FIG. 2 illustrates a method 200 to effectuate presentation of customized content to subjects within integrated applications, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include storing, in electronic storage, taxonomical classifications of individual pieces of content, psychological profiles for subjects, health assessment information of the subjects, and/or other information. Operation 202 may be performed by electronic storage 126, in accordance with one or more implementations.

An operation 204 may include effectuating presentation of a set of the pieces of content within digital environments via integrated applications interacted with by the subject to fulfill an objective related to the subject. Operation 204 may be performed by one or more hardware processors configured by machine-readable instructions including a component that is the same as or similar to presentation component 110, in accordance with one or more implementations.

An operation 206 may include receiving interaction information from the integrated applications that characterizes interactions between the subject and the set of the pieces of content. Operation 206 may be performed by one or more hardware processors configured by machine-readable instructions including a component that is the same as or similar to information receiving component 112, in accordance with one or more implementations.

An operation 208 may include determining, based on the interaction information, impact values of the pieces of content in the set on the objective for the subject over a period of time. Operation 208 may be performed by one or more hardware processors configured by machine-readable instructions including a component that is the same as or similar to impact determination component 114, in accordance with one or more implementations.

An operation 210 may include determining, based on the impact values, correlations between the pieces of content in the set with aspects of the objective. The correlations may include the corresponding impact values. Operation 210 may be performed by one or more hardware processors configured by machine-readable instructions including a component that is the same as or similar to correlation determination component 116, in accordance with one or more implementations.

An operation 212 may include adjusting, based on the correlations, the digital environments of the integrated applications to regularly include the pieces of content from the set that positively impacted the objective and remove the pieces of content from the set that negatively impacted the objective. Operation 212 may be performed by one or more hardware processors configured by machine-readable instructions including a component that is the same as or similar to environment adjusting component 118, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to effectuate presentation of customized content to subjects within integrated applications, the system comprising:
electronic storage configured to store i) taxonomical classifications of individual pieces of content, wherein individual taxonomical classifications include content parameter values for content parameters that define classifications for the individual pieces of content, ii) psychological profiles for subjects, and iii) health assessment information of the subjects, wherein the taxonomical classifications conform to a taxonomy that defines a hierarchical system of the content parameters that facilitate providing the pieces of content with the classifications, wherein the pieces of content are defined by the content parameter values for some or all of the content parameters, wherein the psychological profiles include psychological parameter values for psychological parameters; and
one or more processors configured by machine-readable instructions to:
effectuate presentation of a set of the pieces of content within digital environments via integrated applications interacted with by the subject to fulfill an objective related to the subject;
receive interaction information from the integrated applications that characterizes interactions between the subject and the set of the pieces of content;
determine, based on the interaction information, impact values of the pieces of content in the set on the objective for the subject over a period of time;
determine, based on the impact values, correlations between the pieces of content in the set with aspects of the objective, wherein the correlations include the corresponding impact values; and
adjust, based on the correlations, the digital environments of the integrated applications to regularly include the pieces of content from the set that positively impacted the objective and remove the pieces of content from the set that negatively impacted the objective.

2. The system of claim 1, wherein the correlations determined are between the content parameter values to the content parameters for individual pieces of the content in set and the aspects of the objective.

3. The system of claim 1, wherein the health assessment information includes a current mental state of a given subject, a current emotional state of the given subject, and/or changes in the psychological profile of the given subject.

4. The system of claim 3, wherein the objective is determination of the health assessment information.

5. The system of claim 4, wherein some of the pieces of content in the set prompt a response from the given subject.

6. The system of claim 4, wherein individual pieces of content in the set correlate with the current mental state of the given subject, the current emotional state of the given subject, and/or changes in the psychological profile of the given subject to facilitate determining the health assessment information.

7. The system of claim 6, wherein determining the impact values includes determining frequent interactions with the pieces of content and determining whether the pieces of content affect the determination of the health assessment information.

8. The system of claim 1, wherein the objective is to provide treatment and/or guidance to affect the health assessment information responsive to the health assessment information such that the pieces of content in the set present the treatment and/or guidance, wherein the aspects of the objective include changes in the health assessment information, and wherein determining the impact values includes determining frequent interactions with the pieces of content and determining positive and/or negative changes to the health assessment information.

9. The system of claim 1, wherein the one or more processors are further configured by the machine-readable instructions to receive application usage information and/or session usage information, wherein determining the impact values are based on the application usage information and/or the session usage information.

10. The system of claim 1, wherein the one or more processors are further configured by the machine-readable instructions to store the interaction information to the electronic storage.

11. A method to effectuate presentation of customized content to subjects within integrated applications, the method comprising:
storing, in electronic storage, i) taxonomical classifications of individual pieces of content, wherein individual taxonomical classifications include content parameter values for content parameters that define classifications for the individual pieces of content, ii) psychological profiles for subjects, and iii) health assessment information of the subjects, wherein the taxonomical classifications conform to a taxonomy that defines a hierarchical system of the content parameters that facilitate providing the pieces of content with the classifications, wherein the pieces of content are defined by the content parameter values for some or all of the content parameters, wherein the psychological profiles include psychological parameter values for psychological parameters;
effectuating presentation of a set of the pieces of content within digital environments via integrated applications interacted with by the subject to fulfill an objective related to the subject;
receiving interaction information from the integrated applications that characterizes interactions between the subject and the set of the pieces of content;
determining, based on the interaction information, impact values of the pieces of content in the set on the objective for the subject over a period of time;
determining, based on the impact values, correlations between the pieces of content in the set with aspects of the objective, wherein the correlations include the corresponding impact values; and
adjusting, based on the correlations, the digital environments of the integrated applications to regularly include the pieces of content from the set that positively impacted the objective and remove the pieces of content from the set that negatively impacted the objective.

12. The method of claim 11, wherein the correlations determined are between the content parameter values to the content parameters for individual pieces of the content in set and the aspects of the objective.

13. The method of claim 11, wherein the health assessment information includes a current mental state of a given subject, a current emotional state of the given subject, and/or changes in the psychological profile of the given subject.

14. The method of claim 13, wherein the objective is determination of the health assessment information.

15. The method of claim 14, wherein some of the pieces of content in the set prompt a response from the given subject.

16. The method of claim 14, wherein individual pieces of content in the set correlate with the current mental state of the given subject, the current emotional state of the given subject, and/or changes in the psychological profile of the given subject to facilitate determining the health assessment information.

17. The method of claim 16, wherein determining the impact values includes determining frequent interactions with the pieces of content and determining whether the pieces of content affect the determination of the health assessment information.

18. The method of claim 11, wherein the objective is to provide treatment and/or guidance to affect the health assessment information responsive to the health assessment information such that the pieces of content in the set present the treatment and/or guidance, wherein the aspects of the objective include changes in the health assessment information, and wherein determining the impact values includes determining frequent interactions with the pieces of content and determining positive and/or negative changes to the health assessment information.

19. The method of claim 11, further comprising receiving application usage information and/or session usage information, wherein determining the impact values are based on the application usage information and/or the session usage information.

20. The method of claim 11, further comprising storing the interaction information to the electronic storage.
